(19) United States
(12) Patent Application Publication
(10) Pub. No.: US 2009/0143733 A1
(43) Pub. Date: Jun. 4, 2009

(54) PATIENT CONTROLLED ANALGESIA DEVICE AND METHOD OF ITS USE

(75) Inventors: Timothy Keenan, Denver, CO (US); James E. Grissom, Brighton, CO (US)

Correspondence Address: WILEY REIN LLP, 1776 K. STREET N.W., WASHINGTON, DC, 20006, US

(73) Assignee: The Redesign Company, LLC, Lakewood, CO (US)

(21) Appl. No.: 11/947,614

(22) Filed: Nov. 29, 2007

(51) Int. Cl. A61M 5/142; A61B 5/117

(52) U.S. Cl. 604/151; 713/186

(57) ABSTRACT

A patient controlled analgesia device generally includes: a patient controlled analgesia pump and a biometric authentication system. The biometric authentication system generally includes a biometric pump controller coupled to the patient controlled analgesia pump that activates the patient controlled analgesia pump to deliver analgesia and a biometric switch adapted to receive a biometric input and operably coupled to the biometric pump controller to activate the biometric pump controller. A biometric authentication processor in the biometric pump controller activates the patient controlled analgesia pump to deliver analgesia only upon authenticating the biometric input received at the biometric switch. Optionally, the biometric pump controller also includes a dosing processor that limits delivery of analgesia according to a prescription.

### BACKGROUND OF THE INVENTION

a. Field of the Invention

The instant invention relates to infusing analgesia or like medication into a patient on demand by the patient. In particular, the instant invention relates to a safety device for reducing the risk of administration of such medication by proxy.

b. Background Art

One of the longest-standing objectives of medicine has always been the efficacious alleviation of chronic and incessant pain. It has often been difficult to effectively control such severe pain even though a number of highly effective painkilling medications are widely known. Since it is difficult for a physician to objectively measure pain in a patient, it has remained difficult to accurately control pain in a manner affording consistently helpful relief to the patient.

Dosage of painkilling medication is based on general dosage guidelines which minimize dosage to prevent side effects of the drug. Such general guidelines often provide inadequate assistance since the effects of any particular medication may vary widely between patients. Accordingly, pain is often either overcontrolled or undercontrolled in many patients, with the corresponding side effects of either a sedative effect or an inadequate diminution in the level of pain, respectively.

Patient controlled analgesia (PCA) is a technique for providing pain relieving medication to patients. Most commonly, it refers to intravenous, epidural, or subcutaneous administration of a liquid opioid via a pumping device with the patient having some ability to control the timing and quantity of drug delivery. Pumps currently in use for PCA generally give the clinician two parameters to set when prescribing a given drug for a patient. These include (1) a dose or bolus amount of drug administered whenever the patient presses a button; and (2) a lockout interval that determines how soon after a bolus is administered a second bolus will be delivered if the patient presses the button again. If a patient presses the button before the lockout interval has elapsed, the PCA pump simply ignores the request. The dose and lockout are programmed into the pump for an individual patient and drug combination. The dose is prescribed based on the clinician's assessment of the patient's opioid requirement (depending on weight, habituation, or other factors). The lockout interval is generally set depending on the time to onset of clinical effect of a given drug. The lockout interval is used to prevent a patient from giving himself or herself another bolus before the previous bolus has had a chance to take effect.

In some patients, significantly less painkilling medication is required with PCA pump therapy than with conventional therapies (e.g., oral ingestion and intramuscular injection). When used as prescribed and intended, therefore, the risk of oversedation is significantly reduced. Adverse events can result, however, when family members, caregivers, or clinicians who are not authorized become involved in administering the analgesia for the patient “by proxy” in a well-intentioned effort to keep the patient comfortable (that is, when someone other than the patient pushes the button that triggers delivery of analgesia).

### BRIEF SUMMARY OF THE INVENTION

Accordingly, it is desirable to provide a safety device for use with a PCA pump that reduces the risk of PCA by proxy.

In one embodiment, the present invention provides a patient controlled analgesia device. The device generally includes: a patient controlled analgesia pump; a biometric pump controller coupled to the patient controlled analgesia pump that activates the patient controlled analgesia pump to deliver analgesia; and a biometric switch adapted to receive a biometric input and operably coupled to the biometric pump controller to activate the biometric pump controller. The biometric pump controller includes a biometric authentication processor that activates the patient controlled analgesia pump to deliver analgesia only upon authenticating the biometric input received at the biometric switch. Typically, the biometric pump controller also includes a biometric identifier memory device in which can be stored at least one biometric identifier, such as a fingerprint (e.g., a thumbprint). The biometric authentication processor can then compare the biometric input received at the biometric switch to a biometric identifier stored in the biometric identifier memory device, and dispense analgesia only if the comparison results in an authentication of the received biometric input. The system will generally include a cable through which the biometric switch is coupled to the biometric pump controller.

Optionally, the biometric pump controller also includes a dosing processor that limits delivery of analgesia according to a prescription.

In another embodiment, the present invention is a biometric authentication system for use with a patient controlled analgesia pump. The biometric authentication system generally includes: a biometric pump controller including an interface connectable to a patient controlled analgesia pump and a biometric authentication processor; and a biometric switch operably coupled to the biometric pump controller to activate the patient controlled analgesia pump. The biometric authentication processor causes the biometric pump controller to output an analgesia-dispensing signal from the interface only upon authenticating a biometric input received at the biometric switch.

Also disclosed herein is a method of dispensing analgesia, generally including the steps of: connecting a biometric authentication system to a patient controlled analgesia pump, the biometric authentication system including a biometric pump controller that outputs a signal that causes the patient controlled analgesia pump to dispense analgesia; storing a biometric identifier in the biometric authentication system; receiving a biometric input in the biometric authentication system; authenticating the biometric input against the stored biometric identifier; and if the biometric input is authenticated, outputting the signal that causes the patient controlled analgesia pump to dispense analgesia.

An advantage of the present invention is that it authenticates the identity of the individual pressing the button to trigger delivery of analgesia prior to dispensing medication, thereby reducing the risk of PCA by proxy.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is an illustration of a PCA device according to an embodiment of the present invention.

FIG. 2 is a functional block diagram of a biometric authentication system according to an embodiment of the present invention.

### DETAILED DESCRIPTION OF THE INVENTION

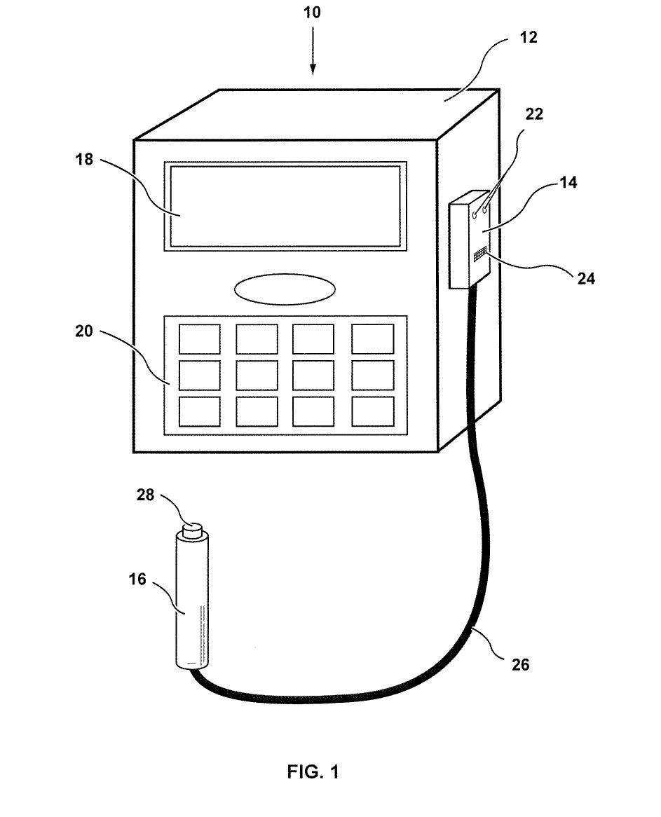

FIG. 1 illustrates a PCA device 10 according to an embodiment of the present invention. PCA device 10 generally includes a PCA pump 12, a biometric pump controller 14, and a biometric switch 16 operably coupled to the biometric pump controller 14. The biometric pump controller 14 and the biometric switch 16 are collectively referred to herein as a biometric authentication system.

As seen in FIG. 1, the PCA pump 12 may include a display screen 18 and a keypad 20. Of course, the PCA pump 12 may also include any other suitable input interface (e.g., a touchscreen). The PCA pump 12 may also include an on/off switch, a replaceable battery, a replaceable drug reservoir cassette, input/output ports, an audible alarm speaker, a replaceable intravenous (IV) tubing, an internal adjustable rate pump, and an internal processor. One of ordinary skill in the art will be generally familiar with the structure, functions, and capabilities of a PCA pump, such that PCA pumps are described herein only to the extent necessary to understand the present invention.

The biometric pump controller 14 is coupled to the PCA pump 12. The biometric pump controller 14 is capable of outputting a signal that causes the PCA pump 12 to deliver analgesia to the patient. The biometric pump controller 14 may also include visual indicators, such as LEDs 22, and a programming button or switch 24, both of which will be described in further detail below.

FIG. 1 depicts an advantageous embodiment of the present invention wherein the biometric authentication system is a stand-alone, plug-and-play device that is externally interfaced with an existing PCA pump. For example, the biometric authentication system may be attached to the PCA pump 12 in lieu of a typical, non-biometric patient control button (that is, the biometric pump controller 14 may be attached to the PCA pump 12 where the patient control button ordinarily would be plugged in). Thus, it is contemplated that a biometric authentication system according to the present invention may be utilized with PCA pumps manufactured by, by way of example only: ALARIS Medical Canada Ltd. of Markham, Ontario; ALARIS Medical Systems Inc. of San Diego, Calif.; Baxter Healthcare Corp. of Round Lake, Ill.; Clinical Resources Inc. of Cockeysville, Md.; Curlin Medical LLC of Huntington Beach, Calif.; Delphi Medical Systems Corp. of Troy, Mich.; First Biomedical Inc. of Olathe, Kans.; Hospira Inc. of Lake Forest, Ill.; MarCal Medical Inc. of Millersville, Md.; McKinley Medical LLP of Wheat Ridge, Colo.; Medical Specialties Distributors Inc. of Stoughton, Mass.; Medtronic MiniMed (Canada) of Mississauga, Ontario; Smiths Medical Canada Ltd. of Markham, Ontario; Smiths Medical MD Inc. of St. Paul, Minn.; and/or Sorenson Medical of Jordan, Utah.

Of course, it is within the spirit and scope of the invention for the biometric pump controller 14 to be internally integrated into the PCA pump 12 (e.g., as part of the internal processor contained within the PCA pump 12), such that only the biometric switch 16 is externally interfaced to the PCA pump 12 in a familiar manner.

The biometric pump controller 14 may be powered through the PCA pump 12 or self-powered, for example via a user-replaceable battery installed in the biometric pump controller 14.

The biometric switch 16 is coupled to the biometric pump controller 14, for example through a flexible multi-conductor cable 26, in order to communicate with the biometric pump controller 14 and activate the PCA pump 12. The biometric switch 16 includes a biometric input device 28, such as a fingerprint scanner, adapted to receive a biometric input, such as a fingerprint, which is preferably a thumbprint.

It is desirable for the biometric switch 16 to be ergonomically designed for hand-held use by a patient, with the patient's thumb resting atop the biometric switch 16 adjacent the biometric input device 28. The biometric input device 28 may, of course, be integrated into an actuator that provides tactile and/or audible feedback, such as a button as would be present on a typical PCA pump switch.

In some embodiments of the invention, the biometric switch 16 receives power from the biometric pump controller 14 and/or the PCA pump 12 through the cable 26, though it is contemplated that the biometric switch 16 may also be self-powered (e.g., via a user-replaceable battery contained within the biometric switch 16).

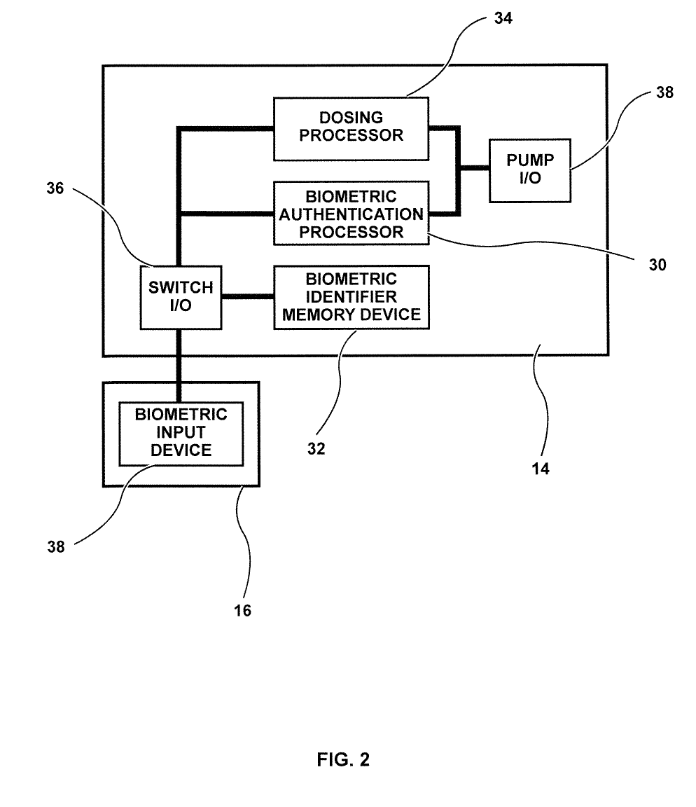

Referring now to FIG. 2, there is shown a functional block diagram of a biometric authentication system according to an embodiment of the present invention. The biometric pump controller 14 typically includes a biometric authentication processor 30 and a biometric identifier memory device 32 (e.g., a non-volatile memory). The biometric pump controller 14 may also include a dosing processor 34, which will be described in further detail below. The term “processor” as used herein refers to a computer microprocessor and/or a software program (e.g., a software module or separate program) that is designed to be executed by one or more microprocessors running on one or more computer systems, analogous hardware-based implementations (e.g., a series of instructions stored in one or more solid-state devices), and combined hardware/software implementations. In some embodiments of the invention, for example, the biometric pump controller 14 contains an electrically erasable programmable read only memory (EEPROM) that contains firmware to control the biometric authentication system.

Of course, the biometric pump controller 14 also includes appropriate input/output interfaces, such as switch I/O interface 36 to communicate with the biometric switch 16 (e.g., to receive a biometric identifier during programming and biometric inputs during use of the PCA device 10) and pump I/O interface 38 to communicate with the PCA pump 12 (e.g., to activate the pump to deliver analgesia).

In use, the biometric authentication system is coupled to the PCA pump 12. For example, the biometric pump controller 14 may be externally coupled to the PCA pump 12 via the pump I/O interface 38. Of course, as described above, it is also contemplated that the PCA pump 12 may include an integrated biometric pump controller.

A biometric identifier (e.g., a thumbprint) is then stored in the biometric identifier memory device 32. Typically, only one biometric identifier will be stored in the biometric identifier memory device 32 at any time, though it is within the spirit and scope of the present invention to store multiple biometric identifiers (e.g., both of the patient's thumbprints) if doing so is desirable. The biometric identifier may be stored by asking the patient to provide an input to the biometric input device 28, for example asking the patient to press his or her thumb down on a biometric fingerprint reader integrated into the biometric switch 16. A clinician can then depress the program button or switch 24, which stores the thumbprint in the biometric identifier memory device 32. Indicators 22 may provide feedback during this process (e.g., a red LED may illuminate if the biometric input device 28 fails to get a good read, and a green LED may illuminate upon successful storage of the thumbprint in the biometric identifier memory device 32). Once the biometric identifier is successfully stored, the PCA device 10 is ready for use.

When analgesia is desired, the patient presses his or her thumb down on the biometric fingerprint reader in a manner similar to how a patient would depress a button in a typical PCA device. This causes the biometric authentication system, and in particular the biometric authentication processor 30, to receive a biometric input. The biometric authentication processor 30 compares the received biometric input to the stored biometric identifier. If the received biometric input matches the stored biometric identifier, the biometric input is authenticated, and the biometric pump controller 14 will output a signal that activates the PCA pump 12 to dispense analgesia. If, however, the received biometric input does not match the stored biometric identifier, the biometric input is not authenticated, and the biometric pump controller 14 will not activate the PCA pump 12 to dispense analgesia. Thus, the present invention advantageously minimizes the likelihood that PCA will be administered by proxy by affirmatively authenticating the identity of the individual requesting the analgesia.

Dosing processor 34 cooperates with biometric authentication processor 30 to ensure that analgesia is dispensed only upon authorized request in accordance with a prescription set by the clinician. That is, dosing processor 34 operates to ensure that any requests, including authorized requests, occurring during the lockout interval, as described above, are ignored, thereby minimizing the likelihood of oversedation. Though FIG. 2 depicts the dosing processor 34 as integrated into the biometric authentication system, and in particular integrated into the biometric pump controller 14, it is within the spirit and scope of the present invention for the dosing processor to be a part of the PCA pump 12 itself.

Although certain embodiments of this invention have been described above with a certain degree of particularity, those skilled in the art could make numerous alterations to the disclosed embodiments without departing from the spirit or scope of this invention. All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail of structure may be made without departing from the spirit of the invention as defined in the appended claims.

What is claimed is:

1. A patient controlled analgesia device, comprising:
a patient controlled analgesia pump;
a biometric pump controller coupled to the patient controlled analgesia pump that activates the patient controlled analgesia pump to deliver analgesia; and
a biometric switch adapted to receive a biometric input and operably coupled to the biometric pump controller to activate the biometric pump controller,
wherein the biometric pump controller includes a biometric authentication processor that activates the patient controlled analgesia pump to deliver analgesia only upon authenticating the biometric input received at the biometric switch.

2. The device according to claim 1, wherein the biometric pump controller further comprises a biometric identifier memory device.

3. The device according to claim 2, wherein the biometric authentication processor compares the biometric input received at the biometric switch to a biometric identifier stored in the biometric identifier memory device.

4. The device according to claim 1, wherein the biometric input is a fingerprint.

5. The device according to claim 1, wherein the biometric pump controller further comprises a dosing processor that limits delivery of analgesia according to a prescription.

6. The device according to claim 1, further comprising a cable, wherein the biometric switch is operably coupled to the biometric pump controller via the cable.

7. A biometric authentication system for use with a patient controlled analgesia pump, the biometric authentication system comprising:
a biometric pump controller comprising:
an interface connectable to a patient controlled analgesia pump; and
a biometric authentication processor; and
a biometric switch operably coupled to the biometric pump controller to activate the patient controlled analgesia pump,
wherein the biometric authentication processor causes the biometric pump controller to output an analgesia-dispensing signal from the interface only upon authenticating a biometric input received at the biometric switch.

8. The system according to claim 7, wherein the biometric input is a fingerprint.

9. The system according to claim 7, wherein the biometric pump controller further comprises a biometric identifier memory device.

10. The system according to claim 7, wherein the biometric pump controller further comprises a dosing processor that limits delivery of analgesia according to a prescription.

11. A method of dispensing analgesia, the method comprising:
connecting a biometric authentication system to a patient controlled analgesia pump, the biometric authentication system including a biometric pump controller that outputs a signal that causes the patient controlled analgesia pump to dispense analgesia;
storing a biometric identifier in the biometric authentication system;
receiving a biometric input in the biometric authentication system;
authenticating the biometric input against the stored biometric identifier; and
if the biometric input is authenticated, outputting the signal that causes the patient controlled analgesia pump to dispense analgesia.
